(19) United States
(12) Patent Application Publication
(10) Pub. No.: US 2023/0202283 A1
(43) Pub. Date: Jun. 29, 2023

(54) DRIVE FACILITY FOR A MEDICAL DEVICE AND MEDICAL DEVICE

(71) Applicant: Siemens Healthcare GmbH, Erlangen (DE)

(72) Inventors: Martin BRAEUER, Nuernberg (DE); Peter NOEGEL, Effeltrich (DE)

(73) Assignee: Siemens Healthcare GmbH, Erlangen (DE)

(21) Appl. No.: 18/146,737

(22) Filed: Dec. 27, 2022

(30) Foreign Application Priority Data
Dec. 29, 2021 (DE) 10 2021 215 074.8

(51) Int. Cl. B60K 6/36; B60K 6/445

(52) U.S. Cl. CPC B60K 6/36; B60K 6/445

(57) ABSTRACT

A drive facility for a medical device comprises a housing, a first motor and a second motor, and a roller facility on the housing and at least partially accommodated in the housing, the roller facility including a roller element, wherein the first motor is configured to pivot the roller facility to pivot the roller element and the second motor is configured to drive the roller element.

### CROSS-REFERENCE TO RELATED APPLICATION(S)

The present application claims priority under 35 U.S.C. §119 to German Patent Application No. 10 2021 215 074.8, filed Dec. 29, 2021, the entire contents of which are incorporated herein by reference.

### FIELD

One or more example embodiments relates to a drive facility for a medical device comprising a housing, a first motor and a second motor, as well as a roller facility arranged on the housing and at least partially accommodated in the housing and comprising a roller element.

### RELATED ART

Medical devices can be embodied as movable so that they can be utilized at different positions. This means that the medical device is not tied to a specific location but can be moved between different locations in order to allow flexible deployment of the device. This imposes high demands in terms of maneuverability in order that the medical device is also able to move in a confined space and be positioned according to the requirements of its use.

Since it is furthermore desirable for medical devices to be able also to move independently in an autonomous or partially autonomous driving mode of operation, it is necessary to implement at least some of the rollers or casters used for the movement also as drivable so that a motor-driven movement of the medical device is possible. There is therefore a need for mechanisms for medical devices which provide for movement of the medical device with a high degree of maneuverability as well as the possibility to be driven.

Conventional steering geometries, such as are known from automotive engineering, for example, are in this case suitable only to a limited extent for medical devices on account of their limited maneuverability. Swivelable casters, as used for example on rollable office chairs, while offering a higher level of maneuverability, are complicated to drive via a motor due to the embodiment of the casters.

### SUMMARY

The object underlying the invention is therefore to disclose an improved drive facility for a medical device which enables both a higher level of maneuverability and a drive means.

According to one or more example embodiments, a drive facility for a medical device includes a housing; a first motor; a second motor; and a roller facility on the housing and at least partially accommodated in the housing, the roller facility including a roller element, wherein the first motor is configured to pivot the roller facility to pivot the roller element and the second motor is configured to drive the roller element.

According to one or more example embodiments, the first motor and the second motor are in fixed positions in the housing or the first motor is in a fixed position in the housing and the second motor is on the roller facility.

According to one or more example embodiments, the first motor and the second motor are coupled to the roller facility by a summing gear train, the first motor and the second motor are configured to drive the roller element via a summed torque of the first motor and the second motor when the first motor and the second motor run at a same rotational speed and the first motor and the second motor are configured to pivot the roller element when the first motor and the second motor run at different rotational speeds.

According to one or more example embodiments, the summing gear train is a planetary gear train.

According to one or more example embodiments, the first motor and the second motor are electric motors.

According to one or more example embodiments, drive facility further includes at least one actuating device configured to at least one of actuate the first motor and the second motor or control rotational speeds of the first motor and the second motor, wherein the actuating device is in the housing.

According to one or more example embodiments, the roller facility has a braking mechanism having at least one brake acting on the roller element.

According to one or more example embodiments, the roller facility comprises an absolute position transducer configured to determine a current orientation of the roller element in relation to the housing.

According to one or more example embodiments, a medical device includes at least one drive facility according to one or more example embodiments.

According to one or more example embodiments, the medical device includes two or more rigid rollers, wherein the rigid rollers are along a common axis.

According to one or more example embodiments, the at least one drive facility is at least two drive facilities, the roller elements at least one of along at least one common axis or along at least one common axis by pivoting the roller elements.

According to one or more example embodiments, the at least two drive facilities is at least three drive facilities, the roller elements arrangeable along at least two different axes.

According to one or more example embodiments, the medical device includes a control device configured to actuate the first motor and the second motor of the at least one drive facility to execute at least one of a straight-ahead travel or a rotation of the medical device around a pivot point.

According to one or more example embodiments, at least one of (i) the at least one drive facility is on an underside of a housing of the medical device or (ii) the at least one drive facility is connected to the housing of the medical device by at least one connecting structure.

According to one or more example embodiments, the at least one connecting structure includes at least one of an angled bracing member or a suspension means or a swing axle.

### BRIEF DESCRIPTION OF THE DRAWINGS

Advantages and details of the present invention will become apparent from the exemplary embodiments described hereinbelow, as well as with reference to the drawings, in which:

FIG. 1 shows an exemplary embodiment of a drive facility according to the invention,

FIG. 2 shows a perspective view of the exemplary embodiment of the drive facility,

FIG. 3 shows a first exemplary embodiment of a medical device according to the invention comprising a drive facility according to the invention,

FIG. 4 shows a second exemplary embodiment of a medical device according to the invention comprising three drive facilities according to the invention,

FIG. 5 shows a third exemplary embodiment of a medical device according to the invention comprising five drive facilities according to the invention,

FIG. 6 shows a fourth exemplary embodiment of a medical device according to the invention, and

FIG. 7 shows a fifth exemplary embodiment of a medical device according to the invention.

### DETAILED DESCRIPTION

A roller facility can be pivoted at least by way of the first motor in order to pivot the roller element and the roller element can be driven at least by way of the second motor.

The roller element of the roller facility is therefore pivotable on the one hand, as a result of which a high degree of maneuverability of a medical device is achieved when the latter comprises one or more of the drive facilities. Furthermore, the roller element is also drivable, thus enabling motorized operation of a medical device coupled to the drive facility.

The arrangement of the first motor for the pivoting action and the second motor for driving the roller element in the housing enables the drive facility to be constructed in a compact design. Furthermore, the motors and at least a part of the roller facility can be encapsulated by the housing, thus resulting in particular in a good level of cleanability that is advantageous for a medical device, in particular with regard to the absence of pathogens which may be necessary for medical reasons.

The drive facility according to one or more example embodiments of the present invention can advantageously be used in different numbers for medical devices in order to enable the medical devices to be electrically movable. In addition to the drive facilities, further non-driven wheels, rollers, casters or similar may also be used in this case, as will be described in greater detail below. The construction of the chassis for a medical device is advantageously simplified as a result since the drive facility is suitable for different types of devices because all the components necessary for providing the movability and maneuverability are arranged in a common housing. The possibility of using the drive facility for different devices has the further advantage that the development costs for the individual medical devices may turn out to be lower since no one-off development for a chassis of the device is required. Furthermore, the drive facility can be developed and manufactured on an industrial scale, thus resulting in reasonable prices for the drive facility owing to the large-volume production achievable thereby.

Compared to steering geometries known from automotive engineering, for example, using a pivotable roller facility has the advantage that very tight curve radii and/or rotations around a pivot point located underneath the medical device are possible. Furthermore, an improved straight-ahead travel can be achieved via the drive facility compared for example to mecanum or omni wheels that can likewise be used for driving medical devices. Moreover, the susceptibility to wear and tear of the drive facility can be reduced owing to the compact design of the drive facility and the small number of parts used.

Advantageously, the drive facility also enables already existing medical devices to be converted to provide an electrical movement capability. The medical device may be for example an imaging device, for example a computed tomography system. Moving smaller devices, for example an ultrasound device, by way of the drive facility is also possible. Furthermore, other devices employed in the medical context, for example for serving meals in the hospital or similar, can be driven by way of the drive facility.

Driving the roller element causes a rolling movement about an axis of rotation of the roller element, thereby enabling a device coupled thereto to operate in a driving mode. The roller element can be implemented for example as a wheel, as a caster or as a comparable element. The pivoting of the roller element is effected in particular around a pivot axis which stands orthogonally to the axis of rotation and in particular can also intersect the axis of rotation, thus enabling a steering movement to be performed as a result of the swiveling of the roller element. In addition to the roller element, the roller facility also comprises in particular a retaining or carrier structure by way of which the roller element is rotatably mounted and which can be pivoted relative to the housing.

In a preferred embodiment of the invention it can be provided that both motors are arranged in fixed positions in the housing or that the first motor is arranged in a fixed position in the housing and the second motor is arranged on the roller facility. The arrangement of the two motors in fixed positions in the housing has the advantage that less mass needs to be moved when pivoting the roller facility. Furthermore, electric cables between the roller facility and the housing can be dispensed with, thus advantageously enabling a 360° pivotability of the roller facility to be realized.

In contrast, the arrangement of the second motor for driving the roller element on the roller facility has the advantage that such an implementation is technically easier to realize since the second motor can be arranged for example in the region of a wheel hub of the roller element such that a direct or mechanically easy-to-realize connection for the transmission of power from the second motor to the roller element is sufficient.

In a preferred embodiment of the invention it can be provided that the motors are coupled to the roller facility by way of a summing gear train, the roller element being drivable via a summed torque of the motors when the motors are running at the same rotational speed and being pivotable via at least a portion of the summed torque when the motors are running at different rotational speeds. In this case the two motors connected to the summing gear train can be arranged in particular in a fixed position in the housing.

The use of a summing gear train, also referred to as a summation gearbox, enables the first motor and the second motor to be used for driving the roller element. The summing gear train enables the summed torque to be used for driving the roller element when both motors are operating at the same rotational speed such that both motors can advantageously be used for the forward propulsion of the device. When the motors are running at different rotational speeds, at least a portion of the summed torque can be used for pivoting the roller element such that a pivoting movement can likewise be implemented by way of the first motor and the second motor. This enables a good utilization of the capacity of the motors since the driving mode, in particular a driving mode comprising operating phases including a straight-ahead mode as well as operating phases including a pivoting of the roller element, can be realized by way of both motors. Advantageously, this enables a higher mechanical driving power to be realized or, as the case may be, smaller motors can be used for a predefined driving power.

According to one or more example embodiments of the present invention it can be provided that the summing gear train is implemented as a planetary gear train. The summing gear train can be arranged inside the housing for example and be coupled on the input side to the first motor and the second motor. On the driven side, the gear train is coupled to the roller facility and the roller element in particular by way of a separate output in each case such that it is possible on the one hand to pivot the roller element via a pivoting of the roller facility and on the other hand to drive the roller element.

The motors are coupled to the roller element via the summing gear train in such a way that when the motors are turning at exactly the same speed, the summed torque of the two motors is transmitted onto the roller element in order to produce a forward movement, whereas differences in the rotational speeds of the motors are converted via the summing gear train into a rotation around the perpendicular axis of the roller facility.

In a preferred embodiment of the invention it can be provided that the motors are implemented as electric motors, in particular electrically commutated and brushless motors, and/or that the motors are each of identical construction.

The use of electrically commutated and brushless electric motors enables in particular a flexible adjustment of the rotational speed of the respective motors and consequently a precise setting of operating states in which both motors run at the same speed or in which the motors run in each case with a specified difference in rotational speed. The use of two motors of identical construction simplifies the actuation of the drive facility or of its motors for a driving mode that is to be implemented by way of the drive facility.

According to one or more example embodiments of the present invention it can be provided that the drive facility comprises at least one actuating device for actuating the motors and/or for controlling their rotational speed, the actuating device being arranged in the housing. The actuating device may be embodied for example for measuring the rotational speeds of the motors, such that the rotational speed can be set in each case by way of a controller, for example. This enables rotational speeds or differences in rotational speed transferred to the actuating device to be set exactly, for example.

It is possible in this case that the actuating device constitutes a common actuating device for the first motor and the second motor or that the first motor and the second motor each have a separate actuating device. The separate actuating devices may in each case be embodied as an inverter which supplies one of the electrically commutated motors with a suitably alternating power voltage in each case. For example, special software runs on the two inverters, which are in particular mechanically and electrically identical, and performs the control of the two motors in such a way that a higher-ranking control device or a higher-ranking vehicle controller in each case exchanges desired and actual values for the speed and the steering angle between the separate actuating devices. Alternatively, the two inverters or their function can be realized in a common actuating device.

Advantageously, a higher-ranking control device of the medical device that is to be driven needs to specify only the desired forward propulsion speed of the roller element and a possibly necessary desired change of the steering angle, i.e. of the angle of the roller element around the vertical axis, to the respective drive facility in order to produce a corresponding movement of the device. This enables different device-side control devices to be easily configured to match the drive facilities used in each case to drive the device or, as the case may be, the drive facilities to be used in a plurality of devices without a reconfiguration of the drive facilities or the motors installed in them and of the actuating device being necessary for this purpose. This advantageously results in a flexible applicability of the drive facility for a multiplicity of different devices.

According to one or more example embodiments of the present invention it can be provided that the roller facility comprises a braking mechanism having at least one brake acting on the roller element. The brake serves as a holding brake for the drive facility or a device coupled to the drive facility. The brake can be embodied as an actuating brake which closes when actuated or as a release brake which opens when actuated. The brake can be held electromagnetically for example and be implemented in such a way that it drops down and blocks the roller element if the supply voltage of the drive facility fails. In this way an unintended movement of the device when selecting the power supply for the drive facility can advantageously be prevented.

In a preferred embodiment of the invention it can be provided that the roller facility comprises an absolute position transducer by way of which the current orientation of the roller element in relation to the housing can be determined. The absolute position transducer therefore enables the position of the roller element in relation to the housing, i.e. the degree of pivoting, or the pivot angle in relation to a reference position of the roller element to be determined.

The absolute position transducer can be embodied for example as an absolute encoder so that the position of the roller element or the pivot angle of the roller element can be determined at any time. Compared to determining the position of the roller element from the previously issued rotation commands, the use of an absolute position transducer has the advantage that the position of the roller element can also be correctly determined when the device is put into operation as well as after service activities or after a failure of the auxiliary energy for the logic of one or more drive facilities of a device.

A medical device according to one or more example embodiments of the present invention comprises at least one drive facility according to the invention. The medical device can be embodied for example as a medical imaging device, for example an X-ray machine such as a computed tomography system. An embodiment of the medical device as an ultrasound device or as some other form of a device that can be used in the everyday clinical routine is also possible.

According to one or more example embodiments of the present invention it can be provided that the device has two or more rigid rollers, the rigid rollers being arranged along a common axis. In this configuration, a drive facility can be arranged for example offset with respect to the axis, thus resulting in the medical device having a three-wheeled structure. This enables the medical device to be rotated about pivot points that lie on the common axis of the rigid rollers. In particular when the roller element of the drive facility is able to pivot through at least 90°, the device can also rotate about points that lie on the axis between the two rigid rollers. Advantageously, it is possible in this case to execute a rotation around a point that is located underneath the medical device, thus achieving a good maneuverability of the device in a confined space.

According to one or more example embodiments of the present invention it can be provided that the device has at least two drive facilities, the rollers of which are arranged along at least one common axis and/or can be arranged along at least one common axis by pivoting the rollers. With two drive facilities, a four-wheeled structure of the device is consequently produced, as a result of which the tipover stability of the device can be improved.

With a combination of two drive facilities having two rigid rollers, the device can likewise be rotated about pivot points that lie on the axis of rotation extending along the rigid rollers. Given a pivotability of the roller element through a sufficiently large angular range, it is also possible to execute a rotation around pivot points that lie between the rigid wheels and consequently are located in particular underneath the device. In this way a rotation of the device can be accomplished with a minimum turning circle, thereby advantageously achieving a good level of maneuverability.

In a preferred embodiment of the invention it can be provided that the device has at least three drive facilities, the rollers of which are arranged or can be arranged along at least two different axes. In particular when no further rigid rollers are used, the use of three or more of the drive modules enables the device to be rotated around any points. This also applies if more than three drive facilities, for example four, five or more drive facilities, are used. This results in a very high level of maneuverability of the device since the possibility of rotating the device around any arbitrary pivot point considerably simplifies a driving mode of the device in particular in narrow environments. The number of drive facilities used can be based in this case for example on the size, the weight and/or the requirements in terms of movement of the device.

According to one or more example embodiments of the present invention it can be provided that the device comprises a control device which is embodied to actuate the motors of the drive facility, the drive facility being actuatable via the control device in order to execute a straight-ahead travel and/or a rotation of the device around a pivot point. In particular when a plurality of drive facilities are used, the rotation of the device around a common pivot point requires different pivoting movements or steering angles to be set for the respective roller elements. In this case, however, depending on the arrangement of the drive facilities relative to one another, there exist certain constraints in order to enable a slip-free driving mode of the device. Advantageously, it is possible via the control device, in which for example the number and the relative arrangement of the drive facilities are stored, to perform a corresponding actuation of the respective drive facilities. The control device can in particular actuate at least one actuating device of the drive facilities in each case, the actuating devices each being embodied for actuating and/or controlling the rotational speed of at least one of the motors, as has been described in the foregoing.

According to one or more example embodiments of the present invention it can be provided that the drive facility is arranged on the underside of a housing of the device and/or that the drive facility is connected to the housing by way of at least one connecting structure. The arrangement of the drive facility on an underside of the housing results in a compact structure of the device since the drive facilities used for moving the device do not project at the sides.

Alternatively, it is possible for the drive facility to be connected to the housing by way of at least one connecting structure. This can in particular cause the drive facility to be arranged laterally offset next to the housing of the device. The tipover stability of the device can be improved as a result, for example. Furthermore, the drive facilities can be arranged by way of the respective connecting structure for example also on devices which do not permit an arrangement of the drive facility under the housing.

According to one or more example embodiments of the present invention it can be provided that the connecting structure is or comprises an angled bracing member and/or that the connecting structure is or comprises a suspension means and/or a swing axle.

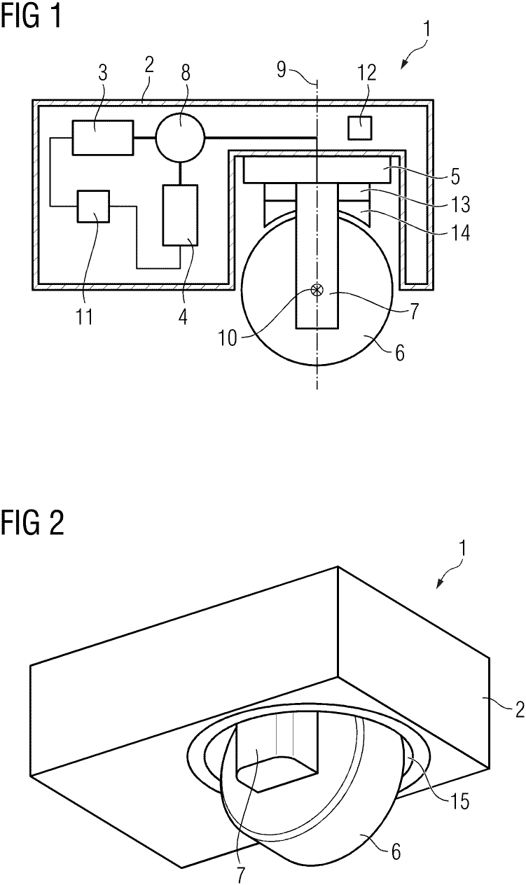

FIG. 1 shows an exemplary embodiment of a drive facility 1. The drive facility 1 comprises a housing 2 as well as a first motor 3 and a second motor 4, which are arranged inside the housing 2. A power supply for the motors 3, 4 can be provided from outside of the drive facility by way of at least one housing-side connection (not shown) of the drive facility 1.

In addition, the drive facility 1 comprises a roller facility 5 which has a roller element 6 embodied as a caster or wheel. The roller element 6 of the roller facility 5 is secured to the housing 2 by way of a carrier structure 7. The roller element 6 can be swiveled relative to the housing 2, the roller element 6 being able to pivot about a vertically extending axis 9. In this case the axis 9 forms the pivot axis or axis of rotation of the swiveling movement of the roller facility 5 or roller element 6. The roller element 6 is furthermore rotatable around a horizontal axis of rotation 10, the axis of rotation 10 of the roller element standing perpendicular to the pivot axis 9 and the two axes 9, 10 intersecting at a common point.

The drive facility 1 further comprises a summing gear train 8 which is coupled to the roller facility 5 as well as to the first motor 3 and the second motor 4. The mechanical connections in FIG. 1 are shown schematically as thick lines.

The first motor 3 and the second motor 4 are in each case implemented as a brushless and electrically commutated electric motor. Further, the first motor 3 and the second motor 4 are each embodied as being of identical construction. In order to drive the motors 3, 4, the drive facility 1 comprises an actuating device 11 which is connected to the motors 3, 4. The electrical connections are drawn schematically in the figure as thin lines. As an alternative to the common actuating device 11, it is also possible to use two separate actuating devices, each assigned to one of the motors 3, 4, by way of which one of the motors 3, 4 can be operated in each case.

The summing gear train 8 is implemented as a planetary gear train. During operation of the motors 3, 4, the roller element 6 is driven by the summing gear train 8 at the same rotational speed via a summed torque of the motors 3, 4. A pivoting of the roller element around the axis of rotation 9 does not take place in this operating state.

In order to effect a pivoting of the roller element 6, the summing gear train is embodied to produce a pivoting of the roller facility 5 and consequently also of the roller element 6 at a different rotational speed of the motors 3, 4 via at least a portion of the summed torque. In order to pivot the roller element 6, an output of the summing gear train 8 is coupled to the carrier structure 7 of the roller facility 5 in such a way that a pivoting of the wheel around the axis 9 is performed. In particular, the roller facility 5 or the roller element 6 can in this case be pivotable through 360°.

The actuating device 11 is embodied to set the rotational speeds of the motors 3, 4 exactly, in particular as part of a rotational speed control function. The actuating device 11 can communicate via an interface (not shown) with a control unit arranged outside of the drive facility 1 and set control commands of the control unit, in particular rotational speeds of the motors 3, 4.

In order to ensure that a correct operation of the drive facility 1 is possible also at an operation startup or after an interruption to operation and/or after an interruption of a power supply of the drive facility 1, for example in the course of troubleshooting and/or service activities, the drive facility 1 additionally comprises an absolute position transducer 12 via which the position of the roller element 6 can be unequivocally determined. The absolute position transducer 12 can be embodied for example as an absolute encoder which outputs the current position of the roller element 3 in relation to the axis 9. Advantageously, the position of the roller element 6 in relation to the axis 9 or in relation to the housing 2 can therefore be established at any point in time.

The drive facility 1 comprises a braking mechanism 13 having at least one brake 14 acting on the roller element 6. The brake 14 can be embodied for example as an actuating brake which closes when the braking mechanism 13 is actuated, or as a release brake which is released when the braking mechanism 13 is actuated. The brake 14 can be held electromagnetically by the braking mechanism 13 for example and be implemented in such a way that it drops down onto the roller element 6 if a supply voltage of the drive facility 1 fails. The brake 14 then blocks a rotational movement of the roller element 6 so that a mechanism coupled to the drive facility 1 can be held stationary.

FIG. 2 shows a perspective view of the drive facility 1. As can be seen, the housing 2 of the drive facility 1 encapsulates the components arranged in the interior of the housing. The roller element 6 at least partially protrudes from an opening 15 of the housing 2 so that a contact of the roller element 6 with a subsurface is possible, as well as a pivoting of the roller element 6 within the opening 15. A mechanical connection that is possibly used for transmitting power from the summing gear train 8 to the roller element 6 can extend for example within the carrier structure 7. The housing 2 advantageously enables the drive facility 1 to be implemented as an easily cleanable component so that when the drive facility 1 is employed as part of a medical device, a possibly necessary absence of pathogens can be achieved.

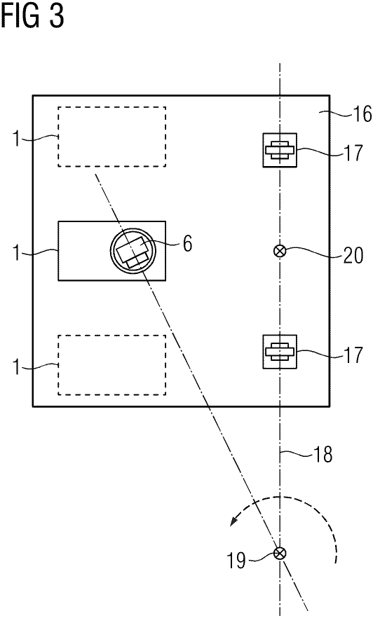

FIG. 3 shows a view of the underside of an exemplary embodiment of a medical device 16. The medical device 16 comprises a drive facility 1 according to the previously described exemplary embodiment. The medical device 16 additionally comprises two rigid rollers 17, which are arranged along a common axis 18.

The medical device 16 can be moved via the electromotive drive of the roller element 6 by way of the motors 3, 4. In order to execute a steering action or a rotation, the roller element 6 can be pivoted as described in the foregoing. Depending on the set pivot angle, a rotation around a pivot point 19 can be performed in the process. Due to the method of construction, said pivot point 19 lies on the axis 18 which connects the rigid rollers 17. In a pivoting movement of the roller element through 90° so that it stands perpendicular to the rigid rollers 17, a rotation of the device 16 can also be performed around a pivot point 20 which lies between the rigid rollers 17 on the axis 18.

It is possible for a second drive facility 1 to be provided in this exemplary embodiment. In this case the first drive facility 1 and the second drive facility 1 can be arranged accordingly at the positions indicated by dashed lines in the drawing, as a result of which the tipover stability of the device 16 can be improved. Even with two drive facilities 1, a rotation of the device 16 around a pivot point 19, 20 lying on the axis 18 can accordingly be performed.

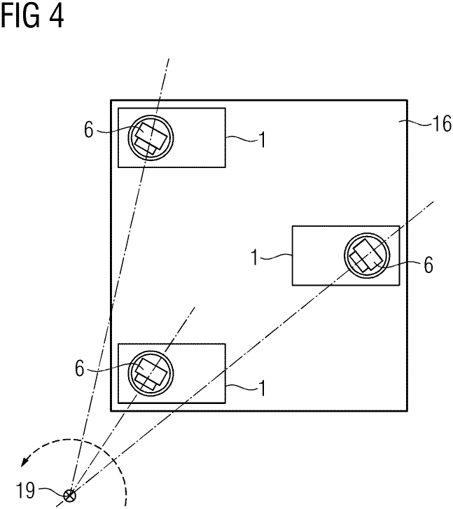

FIG. 4 shows a second exemplary embodiment of a medical device. In this exemplary embodiment, the medical device 16 comprises three drive facilities 1 which are arranged offset relative to one another and can be disposed underneath the device 16 along at least two axes. By using three drive facilities 1 it is possible to rotate the medical device around any arbitrary pivot point 19.

For a chosen pivot point, specific pivot angles result here in each case for the individual roller elements 6, with which a slip-free movement of the device 16 is possible. Said pivot angles requiring to be set can be determined for example by a control device (not shown here) of the medical device 16 as a function of the arrangement of the drive facilities 1 on the medical device 16 and also as a function of a driving maneuver that is to be executed. The control device can then command the actuating devices 11 of the respective drive elements 1 to actuate the respective motors 3, 4 in order to effect the pivoting of the corresponding roller facilities 5. An actuation for a drive of the roller elements 6 so as to perform a movement of the device 16 is also possible by way of the control device.

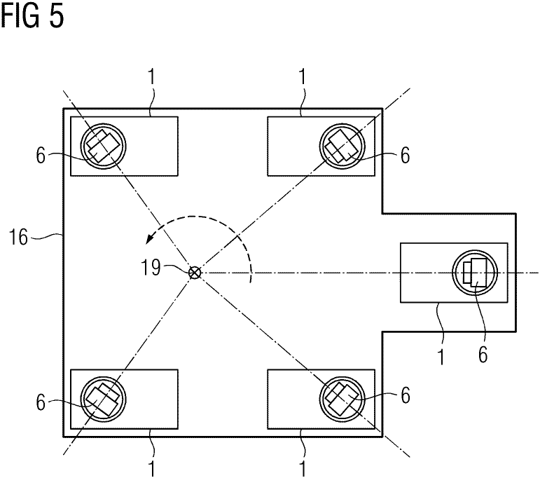

FIG. 5 shows a third exemplary embodiment of a medical device 16. In this exemplary embodiment, the medical device 16 comprises five drive elements 1 via which a rotation of the device 16 around arbitrary pivot points 19 can likewise be accomplished.

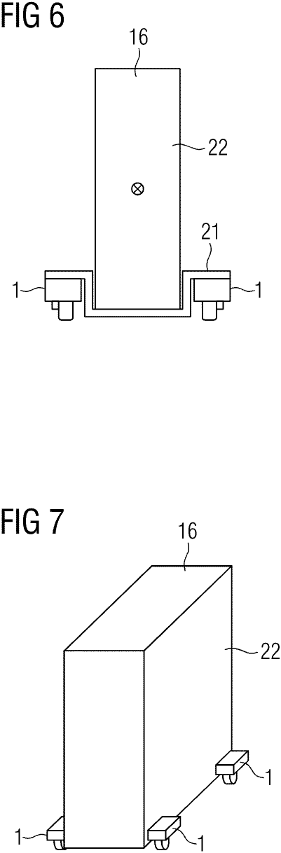

FIG. 6 shows a side view of a fourth exemplary embodiment of a medical device 16. In this exemplary embodiment, the drive facilities 1 are connected to a housing 22 of the device 16 by way of a connecting structure 21. The connecting structure 21 is embodied in this case as an angled bracing member. Alternatively, it is also possible for the connecting structure 21 to be implemented as a suspension means and/or a swing axle or for it to comprise a suspension means and/or a swing axle. The medical device 2 can be moved across the floor at a low height by way of the drive facility 1.

FIG. 7 shows a perspective view of a fifth exemplary embodiment of a medical device 16. In this exemplary embodiment, the medical device 16 comprises four drive facilities 1 which are arranged in each case at the bottom corners of the housing 22. In this arrangement, the drive facilities 1 are secured to the sides of the housing 22 in each case.

In all the preceding exemplary embodiments, a control device of the device 16 can be used in order to perform the actuation of the drive facilities 1 in each case, as has been described in relation to the second exemplary embodiment. The control device can in this case be embodied in particular for performing an autonomous and/or partially autonomous driving mode of the medical device 16.

The motors 3, 4 are embodied in particular as being of identical construction in order to simplify the actuation or setting of the rotational speeds as far as possible. Advantageously, the drive facilities 1 enable these to be arranged in any desired geometry on a medical device 16 according to the latter’s requirements. The actuation can be performed in this case by way of the control device, the number of drive facilities 1 used and their respective arrangement needing to be stored only in the control device so that no changes are required to be implemented in the actuating devices 11. The drive facilities 1 can therefore be used as flexibly deployable drive modules on a great number of different devices 16.

It is possible in all the exemplary embodiments for the drive facility 1 or drive facilities 1 to be implemented without the summing gear train 8. In this case the pivoting of the roller element 6 can be effected by way of the first motor 3 only. The roller element 6 is then driven accordingly by way of the second motor 4 only. A common drive by way of the two motors, as is possible when the summing gear train 8 is used, is omitted in such an exemplary embodiment. When the roller element is driven exclusively by way of the second motor, it is possible for the second motor to be arranged, not in a fixed position in the housing 2 of the drive facility 1, but arranged on the roller facility 5, for example in the region of a hub of the roller element 6.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein and mentioned above, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system’s registers and memories into other data similarly represented as physical quantities within the computer system memories or registers or other such information storage, transmission or display devices.

In this application, including the definitions below, the term ‘module’, ‘interface’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in greater detail on the basis of one or more example embodiments, the invention is not limited by the disclosed examples and other variations may be derived herefrom by the person skilled in the art without leaving the scope of protection of the invention.

1. A drive facility for a medical device comprising:
a housing;
a first motor;
a second motor; and
a roller facility on the housing and at least partially accommodated in the housing, the roller facility including a roller element, wherein the first motor is configured to pivot the roller facility to pivot the roller element and the second motor is configured to drive the roller element.

2. The drive facility of claim 1, wherein the first motor and the second motor are in fixed positions in the housing or the first motor is in a fixed position in the housing and the second motor is on the roller facility.

3. The drive facility of claim 1, wherein the first motor and the second motor are coupled to the roller facility by a summing gear train, the first motor and the second motor are configured to drive the roller element via a summed torque of the first motor and the second motor when the first motor and the second motor run at a same rotational speed and the first motor and the second motor are configured to pivot the roller element when the first motor and the second motor run at different rotational speeds.

4. The drive facility of claim 3, wherein the summing gear train is a planetary gear train.

5. The drive facility of claim 1, wherein the first motor and the second motor are electric motors.

6. The drive facility of claim 1, further comprising:
at least one actuating device configured to at least one of actuate the first motor and the second motor or control rotational speeds of the first motor and the second motor, wherein the actuating device is in the housing.

7. The drive facility of claim 1, wherein the roller facility has a braking mechanism having at least one brake acting on the roller element.

8. The drive facility of claim 1, wherein the roller facility comprises an absolute position transducer configured to determine a current orientation of the roller element in relation to the housing.

9. A medical device comprising:
at least one drive facility, the at least one drive facility including the drive facility of claim 1.

10. The medical device of claim 9, further comprising:
two or more rigid rollers, wherein the rigid rollers are along a common axis.

11. The medical device of claim 9, wherein the at least one drive facility is at least two drive facilities, the roller elements at least one of along at least one common axis or along at least one common axis by pivoting the roller elements.

12. The medical device of claim 11, wherein the at least two drive facilities is at least three drive facilities, the roller elements arrangeable along at least two different axes.

13. The medical device of claim 9, further comprising:
a control device configured to actuate the first motor and the second motor of the at least one drive facility to execute at least one of a straight-ahead travel or a rotation of the medical device around a pivot point.

14. The medical device of claim 9, wherein at least one of (i) the at least one drive facility is on an underside of a housing of the medical device or (ii) the at least one drive facility is connected to the housing of the medical device by at least one connecting structure.

15. The medical device of claim 14, wherein the at least one connecting structure includes at least one of an angled bracing member or a suspension means or a swing axle.

16. The drive facility of claim 2, wherein the first motor and the second motor are coupled to the roller facility by a summing gear train, the first motor and the second motor are configured to drive the roller element via a summed torque of the first motor and the second motor when the first motor and the second motor run at a same rotational speed and the first motor and the second motor are configured to pivot the roller element when the first motor and the second motor run at different rotational speeds.

17. The drive facility of claim 16, wherein the summing gear train is a planetary gear train.

18. The drive facility of claim 16, wherein the first motor and the second motor are electric motors.

19. The drive facility of claim 18, further comprising:
at least one actuating device configured to at least one of actuate the first motor and the second motor or control rotational speeds of the first motor and the second motor, wherein the actuating device is in the housing.

20. The drive facility of claim 19, wherein the roller facility comprises an absolute position transducer configured to determine a current orientation of the roller element in relation to the housing.
